(19) United States
(12) Patent Application Publication
(10) Pub. No.: US 2022/0047615 A1
(43) Pub. Date: Feb. 17, 2022

(54) Immune Supplement Composition

(71) Applicant: Locus IP Company, LLC, Solon, OH (US)

(72) Inventors: Sean FARMER, Ft. Lauderdale, FL (US); Ken ALIBEK, Solon, OH (US); Albina TSKHAY, Solon, OH (US)

(21) Appl. No.: 17/296,330

(22) PCT Filed: Nov. 26, 2019
(86) PCT No.: PCT/US2019/063358

§ 371 Date: May 24, 2021

Related U.S. Application Data
(60) Provisional application No. 62/773,214, filed on Nov. 30, 2018.

(51) Int. Cl. A61K 31/7048; A61K 31/704; A61K 31/23; A61K 33/04; A61K 31/522; A61K 31/52; A61K 31/7056; A61K 31/675; A61K 31/662; A61K 31/7072; A61K 31/7105

(52) U.S. Cl. CPC A61K 31/7048; A61K 31/704; A61K 31/23; A61K 33/04; A61K 31/522; A61K 31/7105; A61K 31/7056; A61K 31/675; A61K 31/662; A61K 31/7072; A61K 31/52

(57) ABSTRACT

The present invention provides compositions and methods for treating a subject infected with a human herpesvirus (HHV). In preferred embodiments, the subject compositions comprise tellimagrandin II, glycyrrhizin, monolaurin, selenium and one or more biological amphiphilic molecules, which, when administered to a subject can support immune health and suppress and/or disable viral pathogenic agents in the body. The invention can also be used for preventing reactivation of a latent HHV and treating and/or preventing diseases, disorders, conditions and/or comorbidities caused by and/or associated with HHV infection.

### CROSS-REFERENCE TO RELATED APPLICATION

This application claims priority to U.S. Provisional Patent Application No. 62/773,214, filed Nov. 30, 2018, which is incorporated herein by reference in its entirety.

### BACKGROUND OF INVENTION

Human herpesviruses (HHVs) are distributed worldwide, with more than 90% of adults infected by one or more HHVs. The HHV family contains three sub-families: the alpha sub-family [herpes simplex virus 1 (HSV-1), HSV-2, and varicella-zoster virus (VZV)], beta sub-family [human cytomegalovirus (CMV), HHV6, and HHV7)], and gamma sub-family [Epstein-Barr virus (EBV) and Kaposi's sarcoma-associated herpesvirus (KSHV)]. These viruses typically establish latent infection in their host, and in certain pathophysiological conditions, undergo lytic reactivation (Lan and Luo 2017). As part of the human virome, an infection with a HHV can impact susceptibility to other infections and disease-causing agents (Sehrawat et al. 2018).

Apart from causing acute infections, such as, for example, cold sores, genital herpes, mononucleosis and shingles, chronic human herpesvirus infections play a role in etiology and pathogenesis of a variety of so-called “non-infectious diseases.” In fact, a large number of diseases and conditions are associated with at least one type of HHV, including, for example, certain cancers, neurologic conditions, lymphatic disorders, systemic and immune system aging, reduced survival rate in the elderly and shortened telomeres.

Despite the prevalence of HHVs and their pathogenicity, there are limited options for the treatment of chronic viral infections. Antivirals such as, for example, Acyclovir, Valacyclovir, Famvir and Gancyclovir can be used to reduce the severity of the active virus, and to suppress future activation. However, due to the limited spectrum of antivirals that are available, HHVs may develop resistance relatively quickly (Gilbert et al. 2002). For example, about 20% of people are infected with antiviral-resistant CMV, and up to 36% of people are infected with antiviral-resistant HSV (Piret and Boivin 2017).

Due to the number of people who are affected by chronic herpetic viral infections, and the lack of reliable long term treatments to suppress and/or cure these viruses, new treatment approaches are needed.

### BRIEF SUMMARY

The present invention provides supplement compositions and methods for enhancing a subject's immune health. Specifically, the present invention is useful for enhancing the immune health of a subject who is infected with a human herpes virus (HHV). Even more specifically, the present invention relates to treating an active HHV infection and/or preventing the reactivation of a latent HHV.

Embodiments of the present invention provide compositions and methods of their use for treating HHV infection and/or preventing reactivation thereof. Advantageously, in some embodiments, the use of naturally-derived components can be more effective than common antiviral medications due to, for example, effectiveness against antiviral-resistant strains and a reduced likelihood of a strain developing resistance.

In certain embodiments, the present invention provides a supplement composition for enhancing the immune health of a subject infected with an HHV, wherein the composition comprises ingredients that help support the subject's immune system and suppress infectious agents in the subject's body. Additionally, the composition can comprise ingredients that are natural or naturally-derived.

In one embodiment, the supplement composition comprises therapeutically-effective amounts of tellimagrandin II, glycyrrhizin, monolaurin and selenium.

In one embodiment the supplement composition comprises, consists of, or consists essentially of therapeutically-effective amounts of tellimagrandin II, glycyrrhizin, monolaurin and selenium.

In certain embodiments, the supplement composition further comprises a biological amphiphilic molecule. In a specific embodiment, the biological amphiphilic molecule is a surfactant, preferably a biosurfactant. Biosurfactants are surface active compounds that lower the surface and interfacial tension between individual molecules at respective surfaces and interfaces. Among other capabilities, biosurfactants provide additional immune support against viral infections, and enhance the bioavailability of the other components of the supplement composition.

In one embodiment, the biosurfactant is a glycolipid produced by, for example, a yeast. In a specific embodiment, the glycolipid is a sophorolipid (SLP). In one embodiment, the biosurfactant is a lipopeptide produced by a bacteria. In a specific embodiment, the lipopeptide is surfactin.

In one embodiment, the biological amphiphilic molecule is a saponin. Saponins are natural surfactants that are found in many plants and that exhibit similar characteristics to microbial biosurfactants.

In one embodiment, the components of the supplement composition are formulated as a mixture, comprising optional additional ingredients, such as, for example, a pharmaceutically-acceptable carrier.

In one embodiment, the supplement composition is formulated into a biosurfactant delivery system, wherein a biosurfactant forms a liposome, or a micro- or nanocapsule, with the supplement composition encapsulated therein. In one embodiment, additional biological polymers can be included to provide further structure for encapsulation.

Biosurfactant encapsulation can enhance the bioavailability of the supplement composition by protecting it from components in the blood, such as proteins and other molecules, that otherwise might bind to the compound and prevent it from penetrating a target site. Additionally, the encapsulated delivery system can allow for compounds that might otherwise be degraded by acids or enzymes in the GI tract to be administered orally, as it creates a barrier against the acids or enzymes. Furthermore, the biosurfactant-encapsulated delivery system formulation allows for time release of the compound(s) therein, thereby reducing the potential toxicity or potential negative side-effects in a subject.

In preferred embodiments, the present invention provides methods for treating an HHV infection and/or preventing reactivation thereof in a subject, wherein the method comprises administering to the subject a therapeutically-effective amount of a supplement composition of the present invention.

In one embodiment, the method can be used to treat and/or prevent a symptom or a comorbidity of an HHV infection, including, for example, cancer or a neurologic disorder.

The subject can be any mammal who is infected with an HHV, whether the virus is latent or active. In certain embodiments, the subject has a compromised immune system. In preferred embodiments, the subject is a human.

The subject can be infected with any HHV, including, for example, one or more of herpes simplex virus 1 (HSV-1), HSV-2, varicella-zoster virus (VZV), cytomegalovirus (CMV), HHV6, HHV7, Epstein-Barr virus (EBV) and Kaposi's sarcoma-associated herpesvirus (KSHV).

In one embodiment, the method first comprises testing the subject for, and/or diagnosing the subject with, an HHV infection. In one embodiment, the testing is performed using known testing methods, including blood antibody tests, and in the case of viruses that cause, for example, external lesions, sampling, culturing and polynucleotide sequencing.

Advantageously, the present invention can lead to simultaneous enhancement in a subject's immune health, improvement of signs, symptoms and comorbidities of an HHV infection, reduction in the number and/or severity of viral reactivations, and reduction in the development of antiviral-resistant strains of HHVs.

### DETAILED DISCLOSURE

The present invention provides supplement compositions and methods for enhancing a subject's immune health. Specifically, the present invention is useful for enhancing the immune health of a subject who is infected with a human herpes virus (HHV). Even more specifically, the present invention relates to treating an active HHV infection and/or preventing the reactivation of a latent HHV.

### Selected Definitions

As used herein, the term “subject” refers to an animal who has a is infected with an HHV, whether the virus is latent or active. The animal may be selected from, for example, pigs, horses, goats, cats, mice, rats, dogs, primates, e.g., apes, chimpanzees and orangutans, guinea pigs, hamsters, cows, sheep, birds, e.g., chickens, reptiles, fish, as well as any other vertebrate or invertebrate. The preferred subject in the context of this invention is a human of any gender and at any age or stage of development, including infant, toddler, adolescent, teenager, adult, middle-aged and senior.

As used herein, “infection” refers to the introduction and/or presence of a disease-causing, or pathogenic agent, into and/or in an organism, tissue or cell. The disease-causing agent can be, for example, a virus, bacteria, fungus, mold, protozoa, prion or parasite.

As used herein, “treating” or “treatment” of a disease, condition or disorder means the eradicating, improving, reducing, ameliorating or reversing of at least one sign or symptom of a disease, condition or disorder (e.g., an infection). Treatment can include, but does not require, a complete cure, meaning treatment can also include partial eradication, improvement, reduction, amelioration or reversal to any degree.

As used herein “preventing” or “prevention” means suppressing, delaying, inhibiting, forestalling, and/or minimizing the onset or progression of a situation or occurrence (e.g., an infection, disease, condition or disorder). Prevention can include reducing the severity of the onset of the situation or occurrence, and/or inhibiting the progression of a situation or occurrence to one that is more severe. Furthermore, prevention can include, but does not require, indefinite, absolute or complete prevention, meaning the situation or occurrence may still develop, but at a later time and/or with lesser severity than it would without preventative measures.

The terms “therapeutically effective” amount or dose, “effective amount,” and “effective dose” are used in this disclosure to refer to an amount of a compound or composition that, when administered to a subject, is capable of providing a desired therapeutic effect (e.g., treatment of an infection) or a desired level of treatment. The actual amount of the compound or composition will vary depending on a number of factors including, but not limited to, the particular disease being treated, the severity of the disease, the size and health of the patient, and the route of administration. A skilled medical practitioner can readily determine the appropriate amount using methods known in the medical arts.

A plant “extract,” as used herein, refers to the material resulting from exposing a plant part to a solvent and removing the solvent, or from using various chemical, immunological, biochemical or physical procedures known to those of skill in the art, including but not limited to, precipitation, steam distillation, centrifugation, filtering, column chromatography, detergent lysis and cold pressing (or expression). Plant extracts can include, for example, essential oils. Plant material can include roots, stems, leaves, flowers, or parts thereof.

As used herein, the term “probiotic” refers to microorganisms, which, when administered in adequate amounts, confer a health benefit on the host. In certain embodiments, a probiotic can be administered to a host's digestives tract to improve the host's digestive health.

The terms “natural” and “naturally-derived,” as used in the context of a chemical compound or substance is a material that is found in nature, meaning that it is produced from earth processes or by a living organism. A natural product can be isolated or purified from its natural source of origin and utilized in, or incorporated into, a variety of applications, including foods, beverages, cosmetics, and supplements. Natural products can be combined with other natural or non-natural products, with which they are not found together in nature. A natural product can also be produced in a lab by chemical synthesis, provided no artificial components or ingredients (i.e., synthetic ingredients that cannot be found naturally as a product of the earth or a living organism) are added.

The terms “isolated” or “purified,” when used in connection with biological or natural materials such as nucleic acid molecules, polynucleotides, polypeptides, proteins, organic compounds, such as small molecules, microorganism cells/strains, or host cells, means the material is substantially free of other compounds, such as cellular material, with which it is associated in nature. That is, the materials do not occur naturally without these other compounds and/or have different or distinctive characteristics compared with those found in the native material.

In certain embodiments, purified compounds are at least 60% by weight the compound of interest. Preferably, the preparation is at least 75%, more preferably at least 90%, and most preferably at least 99% or 100% (w/w) of the desired compound by weight. Purity is measured by any appropriate standard method, for example, by column chromatography, thin layer chromatography, or high-performance liquid chromatography (HPLC) analysis.

The description herein of any aspect or embodiment of the invention using terms such as “comprising,” “having,” “including,” or “containing” with reference to an element or elements is intended to provide support for a similar aspect or embodiment of the invention that “consists of,” “consists essentially of,” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The term “consisting essentially of,” as used herein, limits the scope of the ingredients and steps to the specified materials or steps and those that do not materially affect the basic and novel characteristics) of the present invention.

Use of the term “comprising” contemplates embodiments that “consist” or “consist essentially” of the recited components).

### Supplement Compositions

Embodiments of the present invention provide compositions and methods of their use for treating HHV infections and/or preventing reactivation of a latent HHV in a subject. In one embodiment, the supplement composition comprises one or more of each of the following: a plant-derived compound or extract, a trace mineral and a biological amphiphilic molecule.

Advantageously, in some embodiments, the composition comprises certain natural components, which can be more effective than common antiviral medications due to, for example, greater effectiveness against antiviral-resistant strains and reduced likelihood of a strain developing resistance.

In certain embodiments, the supplement composition comprises ingredients that help support the immune system and suppress and/or disable infectious agents in the subject's body. In one embodiment, the combination of ingredients in the composition creates a synergistic effect towards immune support and/or suppression of infectious agents.

In some embodiments, the supplement composition comprises therapeutically-effective amounts of tellimagrandin II, glycyrrhizin, monolaurin and selenium.

In some embodiments, the supplement composition comprises, consists of, or consists essentially of therapeutically-effective amounts of tellimagrandin 11, glycyrrhizin, monolaurin and selenium. In certain embodiments, the supplement composition further comprises a biological amphiphilic molecule.

In some embodiments, the supplement composition comprises, consists of, or consists essentially of therapeutically-effective amounts of tellimagrandin II, glycyrrhizin, monolaurin, selenium and a biological amphiphilic molecule.

In one embodiment, the composition comprises tellimagrandin II. Tellimagrandin II is an active compound derived from Syzygium aromaticum, the tree with flower buds commonly known as cloves. Additionally, tellimagrandin II can also be extracted from Geum japonicum and some other plants.

This substance is known to be active against HSV and CMV, which have developed acyclovir and phosphonoacetic acid (PAA) resistant strains. In some embodiments, tellimagrandin II contributes to the effectiveness of the supplement composition by reducing viral yields through, for example, inhibition of viral DNA synthesis.

In certain embodiments, the amount of tellimagrandin II in one dosage of the supplement composition is about 20 mg to about 5,000 mg, preferably about 100 mg to about 4500 mg, more preferably about 150 mg to about 3,500 mg.

In one embodiment, the composition comprises glycyrrhizin. Glycyrrhizin is an active compound derived from Glycyrrhiza uralensis and Glycyrrhiza glabra, otherwise known as licorice root. Structurally, glycyrrhizin is a saponin that can be used as an emulsifier in food and cosmetic products.

This substance exhibits activity against viruses including VZV, SARS coronavirus, EBV, HIV-1, hepatitis, and influenza A virus. In some embodiments, glycyrrhizin contributes to the effectiveness of the supplement composition by reducing viral yields and/or inhibiting plaque formation through, for example, interfering with the virus's replication cycle at the attachment, penetration, uncoating and/or particle release stages. Furthermore, glycyrrhizin can have additional therapeutic effects on, for example, the liver where a viral infection has caused liver dysfunction (e.g., hepatitis).

In certain embodiments, the amount of glycyrrhizin in one dosage of the supplement composition is about 5 mg to about 200 mg, preferably about 50 mg to about 100 mg.

In one embodiment, the composition comprises monolaurin, which is the glycerol ester of lauric acid. Laurie acid is an active antiviral and antibacterial substance found in human breast milk, and comprises about 50% of the saturated fat content of coconut oil. When ingested, lauric acid is converted to monolaurin, which is more biologically active than lauric acid.

Monolaurin is active against influenza virus, pneumovirus, Paramyxovirus (Newcastle), Coronavirus (Avian Infectious, Bronchitis virus), herpes simplex I & II, CMV, EBV, HIV, measles, leukemia virus, Simliki forest virus, HPV, Visna virus, Vesicular stomatitits virus, respiratory syncytial virus, Dengue virus (type 1-4), and lymphocytic choriomeningitis.

In some embodiments, monolaurin contributes to the effectiveness of the supplement composition by reducing viral yields through, for example, prevention of viral attachment to susceptible host cells, prevention of viral replication, disruption of the virus lipid bylayer, and/or disintegration of the viral envelope.

Monolaurin provides additional benefits to a subject due to its effectiveness against Gram-positive bacteria, including, e.g., Anthrax, Listeria monocytogenes, Staphylococcus aureus. Groups A, B, F, and G streptococci, Streptococcus agalactiae, Mycobacteria Clostridium perfringens and Gram-negative bacteria, including, e.g., Chlamydia pneumonia, Neisseria gonorrhoeae, Helicobater pylori, Mycoplasma pneumonia, and Vibrio parahaemolyticus; against yeast, fungi and molds, including, e.g., Aspergillus niger, Saccharomyces cerevisiae, Ringworm/Tinea, Malassezia species, Penicillium citrinum, and Candida utilis; and against a number of protozoa, including, e.g., Giardia lamblia.

In certain embodiments, the amount of monolaurin in one dosage of the supplement composition is about 500 mg to about 4,000 mg, preferably about 600 mg to about 3,500 mg, more preferably about 750 mg to about 3,000 mg.

In one embodiment, the composition comprises selenium. Selenium is a trace mineral that can be found in soils, plants and animal tissue. For example, nuts, fresh and saltwater fish, grains, beef and poultry are all sources of selenium when ingested. The selenium can be in an inorganic form (e.g., selenate or selenite), and/or an organic form (e.g., selenomethionine and selenocysteine).

Selenium is an essential mineral for animals, including humans. It is incorporated into selenoproteins, which have a wide range of pleiotropic effects, ranging from antioxidant and anti-inflammatory effects to the production of active thyroid hormone. Selenium also can provide support for cognitive health, reproductive health, and immune health. Accordingly, selenium deficiency has been associated with increased risk of mortality, poor immune function, and cognitive decline.

Selenium deficiency has also been associated with increased viral pathogenesis, in part due to immune dysfunction. Thus, in some embodiments, selenium contributes to the effectiveness of the supplement composition by supporting a subject's immune system in fighting viral invaders.

In certain embodiments, the amount of selenium in one dosage of the supplement composition is about 10 to about 150 μg, preferably about 20 to about 70 μg, more preferably about 30 to about 60 μg.

In preferred embodiments, the supplement composition further comprises a biological amphiphilic molecule. In certain embodiments, the concentration of biological amphiphilic molecule is about 5% or less, preferably about 0.5% to about 2.5%, more preferably about 0.7 to 1.5%. In one embodiment, more than one biological amphiphilic molecule is used in the supplement composition.

In a specific embodiment, the biological amphiphilic molecule is a surfactant, preferably a biosurfactant. Biosurfactants are a structurally diverse group of surface-active substances produced by microorganisms. Biosurfactants are biodegradable and can be produced using selected organisms on renewable substrates. Most biosurfactant-producing organisms produce biosurfactants in response to the presence of a hydrocarbon source (e.g., oils, sugar, glycerol, etc.) in the growing media. Other media components such as concentration of iron can also affect biosurfactant production significantly.

Microbial biosurfactants are produced by a variety of microorganisms, such as, for example, Pseudomonas spp. (P. aeruginosa, P. putida, P. florescens, P. fragi, P. syringae); Flavobacterium spp.; Bacillus spp. (B. subtilis, B. pumillus, B. licheniformis, B. amyloliquefaciens, B. cereus); Wickerhamomyces spp. (e.g., W. anomalus), Candida spp. (e.g., C. albicans, C. rugosa, C. tropicalis, C. lipolytica, C. torulopsis); Rhodococcus spp.; Arthrobacter spp.; Campylobacter spp.; Cornybacterium spp.; Pichia spp. (e.g., P. anomala, P. guilliermondu, P. occidentalis); Starmerella spp. (e.g., S. bombicola); and so on.

All biosurfactants are amphiphiles. They consist of two parts: a polar (hydrophilic) moiety and non-polar (hydrophobic) group. Due to their amphiphilic structure, biosurfactants increase the surface area of hydrophobic water-insoluble substances, increase the water bioavailability of such substances, and change the properties of bacterial cell surfaces.

Biosurfactants accumulate at interfaces, thus reducing interfacial tension and leading to the formation of aggregated micellar structures. The ability of biosurfactants to form pores and destabilize biological membranes permits their use as antibacterial, antifungal, and hemolytic agents. Combined with the characteristics of low toxicity and biodegradability, biosurfactants are advantageous for use in a variety of application, including human health.

Biosurfactants include glycolipids, lipopeptides, flavolipids, phospholipids, fatty acid esters, and high molecular weight polymers such as lipoproteins, lipopolysaccharide-protein complexes, and polysaccharide-protein-fatty acid complexes.

The hydrocarbon chain of a fatty acid acts as the common lipophilic moiety of a biosurfactant molecule, whereas the hydrophilic part is formed by ester or alcohol groups of neutral lipids, by the carboxylate group of fatty acids or amino acids (or peptides), by an organic acid in the case of flavolipids, or, by a carbohydrate in the case of glycolipids.

In one embodiment, the biosurfactants according to the present invention are selected from glycolipids, such as rhamnolipids (RLP), sophorolipids (SLP), trehalose lipids (TL), cellobiose lipids and/or mannosylerythritol lipids (MEL).

In one embodiment, the biosurfactants are selected from lipopeptides, including, for example, surfactin, iturin, fengycin, arthrofactin, viscosin, amphisin and/or lichenysin.

In preferred embodiments, the composition comprises a glycolipid biosurfactant. In a specific embodiment, the glycolipid is a purified SLP. Sophorolipids are glycolipids that comprise a sophorose consisting of two glucose molecules, linked to a fatty acid by a glycosidic ether bond. They can be obtained from fermentation of yeasts, such as Starmerella bombicola.

SLP are categorized into two general forms: the lactonic form, where the carboxyl group in the fatty acid side chain and the sophorose moiety form a cyclic ester bond; and the acidic form, or linear form, where the ester bond is hydrolyzed. In addition to these forms, there exist a number of derivatives characterized by the presence or absence of double bonds in the fatty acid side chain, the length of the carbon chain, the position of the glycosidic ether bond, the presence or absence of acetyl groups introduced to the hydroxyl groups of the sugar moiety, and other structural parameters.

In preferred embodiments, the SLP according to the subject invention are represented by General Formula (1) and/or General Formula (2), and are obtained as a collection of 30 or more types of structural homologues having different fatty acid chain lengths (R³), and, in some instances, having an acetylation or protonation at R¹ and/or R². In certain embodiments, a combination of two or more SLP molecules is utilized.

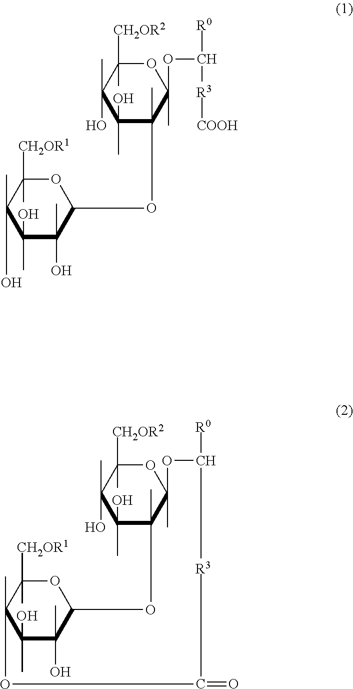

In General Formula (1) or (2), R⁰ can be either a hydrogen atom or a methyl group. R¹ and R² are each independently a hydrogen atom or an acetyl group. R³ is a saturated aliphatic hydrocarbon chain, or an unsaturated aliphatic hydrocarbon chain having at least one double bond, and may have one or more substituents.

Examples of the Substituents include halogen atoms, hydroxyl, lower (C1-6) alkyl groups, halo lower (C1-6) alkyl groups, hydroxy lower (C1-6) alkyl groups, halo lower (C1-6) alkoxy groups, and the like. R³ typically has 11 to 20 carbon atoms, preferably 13 to 17 carbon atoms, and more preferably 14 to 16 carbon atoms. Examples of the halogen atoms or halogen atoms bound to alkyl groups or alkoxy groups include fluorine, chlorine, bromine, and iodine.

SLP have antiviral properties against EBV and HSV, as well as against several pathogenic bacteria, such as Escherichia coli, Moraxella sp., Ralstonia eutropha, Rhodococcus erythropolis, and Salmonella choleraesuis. Additionally, SLP can inhibit microbial quorum sensing and destroy biofllms and/or inhibit their formation. This is particularly useful for treating infections, as biofilm formation by viruses and bacteria allows them to develop resistance to drugs and enhances their pathogenicity.

Thus, in some embodiments, SLP contribute to the effectiveness of the supplement composition through, for example, biofilm disruption and direct antiviral activity. SLP provide additional benefits to a subject, including, for example antibacterial and anti-inflammatory properties.

In certain specific embodiments, the SLP is an acidic form SLP according to General Formula (1). In some embodiments, acidic form SLP comprise greater antiviral properties than other forms of SLP.

In certain specific embodiments, the SLP is a lactonic form SLP according to General Formula (2). In some embodiments, lactonic for SLP comprise greater antibacterial and/or antifungal properties than other forms of SLP.

In some embodiments, the composition comprises a lipopeptide biosurfactant. In a specific embodiment, the lipopeptide biosurfactant is surfactin. Lipopeptides are produced by a variety of probiotics and non-pathogenic bacteria, such as, e.g., Bacillus natto, Bacillus coagulans, Bacillus subtilis, Bacillus amyloliquefaciens, lactic acid bacteria, and others.

Surfactin, in particular, is one of the most powerful lipopeptide biosurfactants. Surfactin is produced by various Bacillus subtilis strains, and has antimicrobial, antitumor, antiviral and antiadhesive properties. It can inhibit fibrin clot formation, induce formation of ion channels in lipid bilayer membranes, and inhibit cyclic adenosine monophosphate (cAMP). In one embodiment, the antiviral activity of surfactin is due to disruption of the viral lipid membrane.

Thus, in some embodiments, surfactin contributes to the effectiveness of the supplement composition through, for example, direct antiviral activity. Surfactin provides additional benefits to a subject, including, for example, antibacterial and anti-cancer properties.

In one embodiment, the biological amphiphilic molecule is a saponin. Saponins are natural surfactants that are found in many plants and that exhibit similar characteristics to microbial biosurfactants, for example, self-association and interaction with biological membranes. There are three basic categories of saponins, including triterpenoid saponins, steroidal saponins, and steroidal glycoalkaloids.

Some well-known triterpenoid saponin-accumulating plant families include the Leguminosae, Amarcmthaceae, Apiaceae, Caryophyllaceae, Aquifoliaceae, Araliaceae, Cucurbitaceae, Berberidaceae, Chenopodiaceae, Myrsinaceae and Zygophyllaceae, among many others, legumes such as soybeans, beans and peas are a rich source of triterpenoid saponins. The steroidal saponins are typically found in members of the Agavaceae, Alliaceae, Asparagaceae, Dioscoreaceae, Liliaceae, Amaryllidaceae, Bromeliaceae, Palmae and Scrophulariaceae families and accumulate in abundance in crop plants such as yam, alliums, asparagus, fenugreek, yucca and ginseng. The steroidal glycoalkaloids are commonly found in members of the Solanaceae family including tomato, potato, aubergines and capsicum.

One notable characteristic of many saponins is their ability to inhibit P-glycoproteins. P-glycoprotein (P-gp) is a member of the ATP-dependent membrane transport proteins and is known to pump substrates out of cells in ATP-dependent mechanisms. The over-expression of P-gp in tumor cells reduces intracellular drug concentrations, which decreases the efficacy of a broad spectrum of antitumor drugs. Accordingly, inhibiting P-gp potentially enhances the cellular bioavailability of some of these compounds.

Thus, in some embodiments, saponins contribute to the effectiveness of the supplement composition by, for example, enhancing the bioavailability of the other compounds present in the composition.

### Formulation and Delivery of the Supplement Compositions

The supplement composition can be formulated to be administered via any route of administration, including, for example, orally, via injection (e.g., intravenous (IV), intramuscular (IM), intraperitoneal, intrathecal or subcutaneous), transdermal, rectal, urogenital (e.g., vaginal), ocular, aural, nasal, inhalation and cutaneous routes.

In one embodiment, the components of the supplement composition are formulated as a mixture, comprising optional additional ingredients, such as, for example, one or more pharmaceutically-acceptable carriers and/or excipients.

The pharmaceutically acceptable carriers and/or excipients, and can be formulated into preparations in, for example, solid, semi-solid, liquid or gaseous forms, such as tablets, capsules, powders, granules, ointments, gels, lotions, solutions, suppositories, drops, patches, injections, inhalants and aerosols.

The term “pharmaceutically acceptable” as used herein means compatible with the other ingredients of a pharmaceutical composition and not deleterious to the recipient thereof.

Carriers and/or excipients according the present invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilisers (such as, e.g., Tween 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatisers, thickeners, coatings, preservatives (such as, e.g., Thimerosal, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like.

In some cases, the carriers can be, for example, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other known carriers appropriate to specific routes of administration. The use of carriers and/or excipients in the field of drugs and supplements is well known. Except for any conventional media or agent that is incompatible with the supplement composition or with, its use in the present compositions may be contemplated.

In one embodiment, the supplement composition is formulated so that it can be delivered to a subject orally. In particular, the composition is formulated as an orally-consumable product.

Orally-consumable products according to the invention are any preparations or compositions suitable for consumption, for nutrition, for oral hygiene or for pleasure, and are products intended to be introduced into the human or animal oral cavity, to remain there for a certain period of time and then to either be swallowed (e.g., food ready for consumption) or to be removed from the oral cavity again (e.g. chewing gums or products of oral hygiene or medical mouth washes). These products include all substances or products intended to be ingested by humans or animals in a processed, semi-processed or unprocessed state. This also includes substances that are added to orally-consumable products (e.g., active ingredients such as extracts, nutrients, supplements, or pharmaceutical products) during their production, treatment or processing and intended to be introduced into the human or animal oral cavity.

Orally-consumable products can also include substances intended to be swallowed by humans or animals and then digested in an unmodified, prepared or processed state. These include casings, coatings or other encapsulations that are intended also to be swallowed together with the product or for which swallowing is to be anticipated.

The orally-consumable product according to the invention can be a composition to be consumed for nutrition or pleasure. These particularly include baked goods (e.g., bread, dry biscuits, cake, cookies, brownies and other pastries), sweets and candies (e.g., chocolates, chocolate bar products, other bar products, gummies, fruit leathers, jelly beans, coated tablets, hard candies, toffees and caramels, and chewing gum), non-alcoholic beverages (e.g., cocoa, coffee, green tea, black tea, herbal teas, lemonades, isotonic beverages, soft drinks, nectars, fruit and vegetable juices, and fruit or vegetable juice preparations), instant beverages (e.g., instant cocoa beverages, instant tea beverages, instant smoothies, instant milkshakes and instant coffee beverages), meat products (e.g., cold cuts, fresh or raw sausage preparations, seasoned oder, marinated fresh meat or salted meat products), eggs or egg products (e.g., dried whole egg, egg whites, and egg yolks), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, ice cream, sherbet, whey, butter, buttermilk, and partly or wholly hydrolyzed products containing milk proteins), products produced from nuts (e.g., nut milks, nut butters, nut flours or powders), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sorbets, fruit smoothies, fruit sauces, and fruit fillings), vegetable preparations (e.g., ketchup, sauces, dried vegetables, deep-freeze vegetables, pre-cooked vegetables, and boiled vegetables), snack articles (e.g., chips, crisps, pretzels, biscuits, crackers and nuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening beverages or other food). The present compositions may also serve as semi-finished products for the production of other compositions intended for nutrition or pleasure.

The composition of the present invention can also be present in the form of capsules, tablets (uncoated and coated tablets, e.g., gastro-resistant coatings), coated tablets, granules, pellets, solid-substance mixtures, dispersions in liquid phases, as emulsions, powders, solutions, pastes or other swallowable or chewable preparations, or as a dietary supplement.

For oral administration, tablets or capsules can be prepared by conventional means with acceptable excipients such as binding agents, fillers, lubricants, disintegrants, or wetting agents. The tablets can be coated, if desired. Preparations for oral administration also can be suitably formulated to give controlled release of the active ingredients. Liquid preparations for oral administration can take the form of, for example, solutions, syrups, or suspensions, or they can be presented as a dry product for constitution with saline or other suitable liquid vehicle before use.

The formulation described herein can also contain acceptable additives as will be understood by one skilled in the art, depending on the particular form of oral delivery. Non-limiting examples of such additives include suspending agents, emulsifying agents, non-aqueous vehicles, preservatives, buffer salts, flavoring, coloring, and sweetening agents as appropriate. Non-limiting examples of specific additives include: gelatin, glycerin, water, beeswax, lecithin, cocoa, caramel, titanium dioxide, and carmine.

In one embodiment, the adjuvant composition can be formulated for administration via injection, for example, as a solution or suspension. The solution or suspension can comprise suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WF1, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Further components can be added to the compositions as are determined by the skilled artisan such as, for example, buffers, carriers, viscosity modifiers, preservatives, flavorings, dyes and other ingredients specific for an intended use. One skilled in this art will recognize that the above description is illustrative rather than exhaustive. Indeed, many additional formulations techniques and pharmaceutically-acceptable excipients and carrier solutions suitable for particular modes of administration are well-known to those skilled in the art. In one embodiment, the supplement composition is formulated into a biosurfactant delivery system, wherein a biosurfactant forms a liposome, or a micro- or nanocapsule, with the supplement composition encapsulated therein. In one embodiment, additional biological polymers can be included to provide further structure for encapsulation.

Biosurfactant encapsulation can enhance the bioavailability of the supplement composition by protecting it from components in the blood, such as proteins and other molecules, that otherwise might bind to the compound and prevent it from penetrating a target site. Additionally, the encapsulated delivery system can allow for compounds that might otherwise be degraded by acids or enzymes in the G1 tract to be administered orally, as it creates a barrier against the acids or enzymes. Furthermore, the biosurfactant-encapsulated delivery system formulation allows for time release of the compound(s) therein, thereby reducing the potential toxicity or potential negative side-effects in a subject.

Methods for Treating, and/or Preventing Reactivation of, HHV Infections

The present invention provides supplement methods for enhancing a subject's immune health. Specifically, the present invention is useful for enhancing the immune health of a subject who is infected with a human herpes virus (HHV). Even more specifically, the present invention relates to treating an active HHV infection and/or preventing the reactivation of a latent HHV.

Advantageously, the present invention can lead to enhancement in a subject's immune health, improvement of signs and symptoms of an HHV infection, reduction in the number and/or severity of viral reactivations, and reduction in the development of antiviral-resistant strains of HHVs. Furthermore, the present invention can lead to improvement and/or prevention of other diseases, disorders, conditions, and/or comorbidities caused by and/or associated with an HHV infection.

In preferred embodiments, the methods of the present invention comprise administering a therapeutically-effective amount of a supplement composition of the present invention to a subject in need thereof.

In certain embodiments, the subject is infected with an HHV. In certain other embodiments, the subject is infected with multiple HHVs. The method can be used as a treatment for infections with HHVs including, for example, herpes simplex virus 1 (HSV-1), HSV-2, herpes zoster, varicella-zoster virus (VZV), cytomegalovirus (CMV), HHV6, HHV7, HHV8, Epstein-Barr virus (EBV) and Kaposi's sarcoma-associated herpesvirus (KSHV). In certain preferred embodiments, the subject is infected with one or more of HSV-1/2, VZV, EBV and CMV.

In some embodiments, the subject is infected with one or more HHVs and/or one or more additional viruses, such as, for example, rubella virus, measles virus, BK virus, JC virus, simian virus (SR40), yellow fever, hepatitis, poliovirus, influenza, pneumovirus, paramyxovirus, coronavirus, HIV, leukemia virus, Simliki forest virus, HPV, Visna virus, vesicular stomatitits virus, respiratory syncytial virus, Dengue virus, or lymphocytic choriomeningitis.

In certain embodiments, the supplement composition can be used for enhancing the immune health of a subject infected with an HHV, wherein the composition comprises ingredients that, for example, help support the immune system and suppress and/or disable infectious agents in the subject's body.

As used herein, “supporting” the immune system can include boosting, improving, enhancing, and/or maintaining the proper functioning of the immune system. Immune support can include support for the cells, tissues, and organs that contribute to proper functioning of the immune system, for example, the lymphatic system, spleen, bone marrow, or any other system involved in production of entities (e.g., antibodies, lymphocytes, red blood cells, white blood cells, platelets) that ward off foreign substances (e.g., inoculants such as viruses) from the body's normal and healthy tissues. Immune support can further include support for parts of the body that aid in preventing and healing from injury, inflammation, cancer, or other non-infectious diseases, ailments, or conditions.

In preferred embodiments, the methods comprise administering a therapeutically-effective amount of a supplement composition comprising therapeutically-effective amounts of tellimagrandin II, glycyrrhizin, monolaurin and selenium. In certain embodiments, the supplement composition further comprises one or more biological amphiphilic molecules.

In some embodiments, the supplement composition comprises, consists of, or consists essentially of therapeutically-effective amounts of tellimagrandin II, glycyrrhizin, monolaurin and selenium.

In some embodiments, the supplement composition comprises, consists of, or consists essentially of therapeutically-effective amounts of tellimagrandin II, glycyrrhizin, monolaurin, selenium and one or more biological amphiphilic molecules.

In one embodiment, the method first comprises testing the subject for, and/or diagnosing the subject with, an HHV infection. In one embodiment, the testing is performed using known testing methods, including blood antibody tests, and in the case of viruses that cause, for example, external lesions, sampling, culturing and polynucleotide (e.g., DNA) sequencing.

In one embodiment, the method further comprises performing follow-up tests on the subject to determine whether, and/or to what extent, the infection has been treated. The subject can be monitored throughout the course of treatment, for example, every day or every other day, in order to determine the status of the infection and whether or not the method is effectively treating the infection. This can include, for example, performing tests, such as those used for diagnosing viral infections, as well as observing the subject for signs of improving health. If follow-up tests show that the rate of improved health is below that which is desired, the dosage of the composition can be adjusted as determined by the skilled practitioner.

In preferred embodiments of the present invention, administration of the supplement composition occurs daily for several days or weeks. Administration can include any known method of drug administration, including, but not limited to, oral, nasal, cutaneous (e.g., applying it as a cream), or intravenous administration.

In one embodiment, the supplement composition is administered to the subject once, twice, or three times per day, determined on a subject-by-subject basis by a skilled physician. Factors to be considered when determining the number of doses to administer include the age of the individual receiving treatment, whether the subject is or may be pregnant (if the subject is female) and the severity of the subject's symptoms In certain embodiments, the present methods can be used to treat an HHV infection in a subject, as well as prevent a latent HHV infection form reactivating in a subject.

The methods of the present invention can be utilized alongside traditional antiviral treatments as a supplement thereto. For example, in some embodiments, the methods can further comprise administering an antiviral drug to the subject, wherein the antiviral is selected from valacyclovir, acyclovir, famciclovir, ganciclovir, valganciclovir, ribavirin, brivudin, cidofovir, fomivirsen, foscamet, penciclovir, vidarabine and others used to treat chronic, congenital, persistent, latent, dormant, acute and/or subacute viral infections.

In one embodiment, the methods can be used to treat and/or prevent the signs and symptoms of HSV-1 and HSV-2 infection, including, for example, sores, vesicles, ulcers, and/or lesions on/in the face, mouth, genitals or rectum, malaise, fatigue, fever, swollen glands, headache, painful urination, discharge and itching.

In one embodiment, the methods can be used to treat and/or prevent the signs and symptoms of EBV infection, including, for example, fatigue, fever, loss of appetite, rash, sore throat, swollen glands, weakness, soreness, and mononucleosis.

In one embodiment, the methods can be used to treat and/or prevent the signs and symptoms of VZV infection, including, for example, rash, blisters, burning, pain, itching, and paresthesia of the skin, fever, fatigue, malaise, loss of appetite, headache, and shingles (when reactivated).

In one embodiment, the methods can be used to treat and/or prevent the signs and symptoms of CMV infection, including, for example, fever, sore throat, swollen glands, diarrhea, GI ulcers, GI bleeding, breathlessness, pneumonia, hypoxemia, mouth ulcers, impaired vision, hepatitis, encephalitis, seizures, coma, hearing loss, jaundice, splotchy skin, enlarged spleen and mononucleosis.

The methods can also be used to treat and/or prevent other diseases, disorders, conditions and/or comorbidities caused by and/or associated with one or more HHV infections. These can be, for example, oncological, cardiovascular/cerebrovascular, neurological, digestive, hepato-pancreatic, autoimmune, urogenital, respiratory, musculoskeletal, endocrine, lymphatic, integumentary, ocular, immune, aging, and psychiatric diseases, disorders, conditions and/or comorbidities.

In one embodiment, the present methods can be used to treat and/or prevent the occurrence of HHV-associated oncological diseases, disorders, conditions and/or comorbidities, including, for example, head and neck cancers, oral cancer, vaginal cancer, cervical carcinoma, breast cancer, colon cancer, testicular cancer, bladder cancer, prostate cancer, pancreatic cancer, rectal cancer, post-transplant lymphoproliferative disease, non-Hodgkin's lymphoma, nasopharyngeal cancer, neck squamous cell carcinoma, stomach cancer, Burkitt's lymphoma, pancreas adenocarcinoma, colon adenocarcinoma, rectum adenocarcinoma, glioblastoma, medulloblastoma, malignant glioma, skin cancer, Kaposi's sarcoma, extranodal lymphoid cancers, CNS cancers, leukemia, and lymphoma.

In one embodiment, the present methods can be used to treat and/or prevent the occurrence of HHV-associated cardiovascular/cerebrovascular diseases, disorders, conditions and/or comorbidities, including, for example, atherosclerosis, acute coronary syndrome, insular cortex lesion, myocardial calcification, stroke, hemorrhagic stroke, transient ischemic attack, myocarditis, inflammatory cardiomyopathy, cardiomyopathy, arterial stiffness, corneal endothelitis, Kawasaki disease, and cutaneous vascular lesions.

In one embodiment, the present methods can be used to treat and/or prevent the occurrence of HHV-associated neurological diseases, disorders, conditions and/or comorbidities, including, for example, autism, Alzheimer's disease, Parkinson's disease, encephalitis, Cerebral palsy, parenchymal lesions, CNS damage, Ramsay Hunt syndrome, multiple sclerosis, encephalopathy, meningitis, febrile seizures, epilepsy, ADHD, chronic fatigue syndrome, amnesia and insomnia.

In one embodiment, the present methods can be used to treat and/or prevent the occurrence of HHV-associated digestive and/or hepato-pancreatic diseases, disorders, conditions and/or comorbidities, including, for example, colitis, hepatic calcification, acute viral diarrhea, liver failure, inflammatory bowel disease, Hodgkin's disease, hepatitis and autoimmune hepatitis.

In one embodiment, the present methods can be used to treat and/or prevent the occurrence of HHV-associated autoimmune diseases, disorders, conditions and/or comorbidities, including, for example, diabetes, Hashimoto's thyroiditis, sepsis, septic shock, focal vasculitis, Guillain-Barre syndrome, rheumatoid arthritis, systemic lupus, erythematosus, exanthemasubitum, Sjögren's syndrome, allergies, and eosinophilic drug allergy.

In one embodiment, the present methods can be used to treat and/or prevent the occurrence of HHV-associated urogenital diseases, disorders, conditions and/or comorbidities, including, for example, infertility, urinary retention, genital lesions, dysuria, cystitis, and kidney allograft infection. In one embodiment, the present methods can be used to treat and/or prevent the occurrence of HHV-associated respiratory diseases, disorders, conditions and/or comorbidities, including, for example, viral pneumonia, acute respiratory infection, asthma, allergic rhinitis, emphysema, pharyngitis, bronchitis, lower respiratory tract infections, idiopathic pulmonary fibrosis, COPD, and emphysema.

In one embodiment, the present methods can be used to treat and/or prevent the occurrence of HHV-associated musculoskeletal diseases, disorders, conditions and/or comorbidities, including, for example, carpal tunnel syndrome, brachial plexus lesion, osteoporosis, musculoskeletal pain, small muscle tumor, frailty, and bone marrow dysplasia.

In one embodiment, the present methods can be used to treat and/or prevent the occurrence of HHV-associated endocrine and/or lymphatic diseases, disorders, conditions and/or comorbidities, including, for example, thymic dysplasia, lymphoepithelioma of the thymus, lymphoid cell necrosis, thymic atrophy, growth retardation, adrenal insufficiency, prostatitis, lymphedema, lymphoproliferative disease, Rosai-Dorfman disease, body-cavity-based non-Hodgkin's lymphomas, Castleman's disease, AILD, and reactive lymphadenopathy.

In one embodiment, the present methods can be used to treat and/or prevent the occurrence of HHV-associated integument and/or ocular diseases, disorders, conditions and/or comorbidities, including, for example, cataract, uveitis, shingles, atopic dermatitis, decreased collagen density, panuveitis, collagen vascular disease, lichen planus, Bowen's disease, and actinic keratosis.

In one embodiment, the present methods can be used to treat and/or prevent the occurrence of HHV-associated aging and/or immune diseases, disorders, conditions and/or comorbidities, including, for example, virus-induced death, senescence, age-related lymphoproliferative disorder, telomere shortening, and general decrease of elderly survival rate.

In one embodiment, the present methods can be used to treat and/or prevent the occurrence of HHV-associated psychiatric diseases, disorders, conditions and/or comorbidities, including, for example, depression (including major depression and psychotic depression), schizophrenia, aggression, and bipolar disorder.

### REFERENCES

Gilbert, C., Bestman-Smith, J., & Boivin, G. (2002). Resistance of herpesviruses to antiviral drugs: clinical impacts and molecular mechanisms. Drug Resistance Updates, 5(2), 88-114. doi: 10.1016/s 1368-7646(02)00021-3. (“Gilbert et al. 2002”).
Lan, K., & Luo, M.-H. (2017). Herpesviruses: epidemiology, pathogenesis, and interventions. Virologica Sinica, 32(5), 347-348. doi: 10.1007/s 12250-017-4108-2. (“Lan and Luo 2017”).
Piret, J., & Boivin, G. (2017). Herpesvirus Resistance to Antiviral Drugs. Antimicrobial Drug Resistance, 1185-1211. doi: 10.1007/978-3-319-47266-9_24. (“Piret and Boivin 2017”).
Sehrawat, S., Kumar, D., & Rouse, B. T. (2018). Herpesviruses: Harmonious Pathogens but Relevant Cofactors in Other Diseases? Frontiers in Cellular and Infection Microbiology, 8. doi: 10.3389/fcimb.2018.00177eH (“Sehrawat et al. 2018”).

1. A supplement composition comprising tellimagrandin II, glycyrrhizin, monolaurin, and selenium, wherein the composition further comprises a biological amphiphilic molecule.

2. (canceled)

3. The composition of claim 2, wherein the biological amphiphilic molecule is a glycolipid biosurfactant, a lipopeptide biosurfactant and/or a saponin.

4. The composition of claim 3, wherein the glycolipid biosurfactant is a sophorolipid (SLP).

5. The composition of claim 3, wherein the lipopeptide biosurfactant is a surfactin.

6. The composition of claim 3, wherein the saponin is a triterpenoid saponin, steroidal saponin and/or steroidal glycoalkaloid.

7. The composition of claim 3, consisting essentially of tellimagrandin II, glycyrrhizin, monolaurin, selenium and one or more biological amphiphilic molecules selected from SLP, surfactin and saponins.

8-9. (canceled)

10. A method for treating a human herpesvirus (HHV) infection and/or preventing reactivation of a latent HHV infection in a subject, wherein the method comprises administering a therapeutically-effective amount of a supplement composition of claim 1.

11. The method of claim 10, further comprising administering a biological amphiphilic molecule to the subject.

12. The method of claim 11, wherein the biological amphiphilic molecule is a glycolipid biosurfactant, a lipopeptide biosurfactant and/or a saponin.

13. The method of claim 10, wherein the supplement composition is administered to the subject orally.

14. The method of claim 10, further comprising diagnosing the subject with one or more HHV infections, and/or performing follow-up tests on the subject to determine whether, and/or to what extent, the HHV infection has been treated.

15. (canceled)

16. The method of claim 10, wherein the infection is caused by one or more of herpes simplex virus 1 (HSV-1), HSV-2, herpes zoster, varicella-zoster virus (VZV), cytomegalovirus (CMV), HHV6, HHV7, HHV8, Epstein-Barr virus (EBV) and Kaposi's sarcoma-associated herpesvirus (KSHV).

17-18. (canceled)

19. The method of claim 10, used to treat and/or prevent a sign or symptom of HSV-1 and/or HSV-2, wherein the sign or symptom is selected from sores, vesicles, ulcers, and/or lesions on/in the face, mouth, genitals or rectum, malaise, fatigue, lever, swollen glands, headache, painful urination, discharge and itching.

20. (canceled)

21. The method of claim 10, used to treat and/or prevent a sign or symptom of EBV, wherein the si tin or symptom is selected from fatigue, fever, loss of appetite, rash, sore throat, swollen glands, weakness, soreness, and mononucleosis.

22. (canceled)

23. The method of claim 10, used to treat and/or prevent a sign or symptom of VZV, wherein the sign or symptom is selected from rash, blisters, burning, pain, itching, and paresthesia of the skin, fever, fatigue, malaise, loss of appetite, headache, and shingles.

24. (canceled)

25. The method of claim 10, used to treat and/or prevent a sign or symptom of CMV, wherein the sign or symptom is selected from fever, sore throat, swollen glands, diarrhea, GI ulcers, GI bleeding, breathlessness, pneumonia, hypoxemia, mouth ulcers, impaired vision, hepatitis, encephalitis, seizures, coma, hearing loss, jaundice, splotchy skin, enlarged spleen and mononucleosis.

26-28. (canceled)

29. The method off claim 10, further comprising administering an antiviral drug used to treat chronic, congenital, persistent, latent, dormant, acute and/or subacute viral infections to the subject, wherein the antiviral is selected from valacyclovir, acyclovir, famciclovir, ganciclovir, valganciclovir, ribavirin, brivudin, cidofovir, fomivirsen, foscamet, penciclovir, and vidarabine.
